(19) United States
(12) Patent Application Publication
(10) Pub. No.: US 2022/0093275 A1
(43) Pub. Date: Mar. 24, 2022

(54) SYSTEMS AND METHODS FOR A MULTI-MODAL GEOGRAPHIC INFORMATION SYSTEM (GIS) DASHBOARD FOR REAL-TIME MAPPING OF PERCEIVED STRESS, HEALTH AND SAFETY BEHAVIORS, FACILITIES DESIGN AND OPERATIONS, AND MOVEMENT

(71) Applicant: Arizona Board of Regents on Behalf of the University of Arizona, Tucson, AZ (US)

(72) Inventors: Esther Sternberg, Tucson, AZ (US); Bo Yang, Tucson, AZ (US); Shujuan Li, Tucson, AZ (US); Matthias Mehl, Tucson, AZ (US); Saurabh Jain, Tucson, AZ (US); Bijoy Dripta Barua Chowdhury, Tucson, AZ (US); Md Tariqul Islam, Tucson, AZ (US); Young-Jun Son, Tucson, AZ (US); Altaf Engineer, Tucson, AZ (US); Philip Stoker, Tucson, AZ (US); Yijie Chen, Tucson, AZ (US)

(21) Appl. No.: 17/479,792

(22) Filed: Sep. 20, 2021

Related U.S. Application Data
(60) Provisional application No. 63/080,594, filed on Sep. 18, 2020.

(51) Int. Cl. G16H 50/80; G16H 50/30; G16H 50/20

(52) U.S. Cl. CPC G16H 50/80; G16H 50/20; G16H 50/30

(57) ABSTRACT

Systems and methods are disclosed for spatially and temporally mapping safer and riskier and wellbeing areas based on perceived safety/risk and wellbeing, related to perceived viral mitigating health behaviors, known built environment wellbeing features, and predictively modelling movement through locations, and building environment features and operations contributing to viral spread.

### CROSS-REFERENCE TO RELATED APPLICATIONS

This application claims the benefit of U.S. Provisional App. No. 63/080,594, filed Sep. 18, 2020, the entire contents of which are incorporated herein by reference.

### FIELD OF THE INVENTION

The present invention, DASH-SAFE (DASHboard-Stress At-Risk Facilities Environment) relates to computer-implemented systems and methods for real-time surveillance, analysis, and mapping of populations at risk of diseases such as COVID-19 using a consolidated technological platform. Post-COVID, it will also be used as an emotion-mapping tool for occupant experience of positive emotions, to map preferred or wellbeing spaces, and in this context will be called DASH-Well.

### BACKGROUND OF THE INVENTION

Diseases like COVID-19 have created significant viral spread and stress among clustered populations that are required to interact in physical locations, like university campuses or similar campus-like environments, e.g. senior living systems, jails, prisons, residential treatment facilities etc. Disease transmission, contact tracing, and mitigation of infection spread are difficult to manage when it is difficult to track population movement and interaction. Moreover, it is difficult to determine, in real-time, events that increase the risk of disease transmission, such as lack of masks, pinch-points, and crowding, inadequate building design and facilities operations, such as toilet plumes, inadequate ventilation, and lack of operable windows. In addition, many elements of the built environment can also enhance wellbeing and resilience and resistance to infection. The DASH-SAFE tool can also be used to help occupants identify spaces that promote wellbeing (e.g. circadian light, spaces to gather, greenery, outdoor spaces, optimal temperature and humidity, spaces to gather safely, spaces for quiet contemplation/spiritual activity, healthy nutrition). This application can be used post-COVID to collect real-time/real-place data on user experience for wellbeing, to help building and outdoor spaces owners/operators and occupants optimally design and use spaces to support wellbeing, and in that context would be called DASH-Well.

As such, there is a need in the art for a system that will spatially and temporally map safer and riskier areas based on perceived safety/risk related to perceived viral mitigating health behaviors, wellbeing spaces, and to predictively model movement through locations, and building environment features and operations contributing to viral spread.

### SUMMARY OF THE INVENTION

In certain embodiments, the present invention comprises:

(1) Creating a real-time interactive GIS-based (Geographic Information System-based) dashboard that will spatially and temporally map safer and riskier areas based on perceived safety/risk related to perceived viral mitigating health behaviors; wellbeing spaces; predictive modelling of movement through campus-like or building environments, e.g. senior living systems, jails, prisons, residential treatment facilities etc.; and built environment features and operations contributing to viral spread or to wellbeing;

(2) Creating an automated alarm system that will signal facilities managers re hot-spots to be addressed;

(3) Creating a navigation tool to provide users safer navigation routes through campus or navigation routes to wellbeing spaces; and

(4) Linking the dashboard with other dashboards tracking proximity of positive cases using WiFi nodes and identifying viral symptoms (e.g., AZ COVID-Watch).

The invention is designed to accomplish such goals as:

(1) Providing a personal risk-assessment and risk management and navigation tool to members of clustered populations in campus-like environments.

(2) Reducing the risk for viral spread and accompanying stress, and providing safer routes to navigate in real-time, by providing real-time information to inform clustered at-risk population users of hot-spots to be avoided as they navigate a campus-like environment.

(3) Providing facilities managers information about hot-spots that need to be addressed to reduce viral spread, without adding to their surveillance burden;

(4) Providing facilities managers and building operators information regarding preferred wellbeing spaces.

(5) Allowing spatial identification of hot spots contributing to infection, by linking to health Apps tracking cases and symptoms, further allowing informed decision-making to mitigate hot-spots and prevent viral spread;

(6) Expanding the capacity and regional placement of existing technologies and contributing significantly to the current national efforts to curb the COVID-19 pandemic; and

(7) By combining and applying existing multi-modal approaches and technologies, the DASH-SAFE/DASH-Well dashboard will enhance their usability, accessibility, and spatial and temporal accuracy for predicting, detecting, and mitigating hot-spots with potential for viral spread.

The present invention addresses an important and extremely urgent problem related to the COVID-19 pandemic, that is: the need for monitoring and modifying both perceived safety/risk related to health prevention behaviors and aspects of the physical environment that contribute to spread of the virus. It addresses an urgent need during post-COVID re-entry to much emptied office spaces, by providing building owners, operators, occupants information about wellbeing spaces to attract occupants and employees to re-enter the workplace. By using a real-time interactive GIS-based dashboard, users will view spatial and temporal information that will help them navigate clustered environments safely and will thus both reduce viral spread and reduce their stress, and will help them identify and find wellbeing spaces within and outside of buildings. The prior research technologies and methods that serve as the key supports for successful creation of the DASH-SAFE/DASH-Well dashboard, have a long track-record of rigorous and successful application in other fields. Successful completion of the aims will accelerate and make it possible to rapidly implement more accurately targeted preventative interventions for control of COVID-19 spread and the stress related to it. Because this is a web-based platform, which can be accessed on any device, it is highly scalable and can be quickly applied to any location where clustered populations occur. This new proposed technology will therefore contribute significantly to the current national efforts to curb the COVID-19 pandemic, and its associated mental health pandemic that has followed in the wake of the viral pandemic, both in terms of viral spread and associated stress, in other campus-like clustered population environments e.g., senior living systems, urban areas, shopping malls, sports arenas, office and industrial parks, military bases, jails, prisons, residential treatment facilities etc.)

The invention comprises a multimodal dashboard that will receive inputs from multiple surveillance technologies, and map onto an interactive GIS campus map to show safer and riskier areas for COVID-re-entry, initially developed at the University of Arizona (UArizona) campus as a test environment for the prototype. The dashboard consists of three components overlaid on an interactive Geographic Information System (GIS) map of the university campus: (1) a joint event-contingent and location-contingent experience sampling survey of perceived safety and risk; (2) predictive modeling of movement of people through campus over time; and (3) facilities management information for aspects of the built environment that are known to relate to risk of viral spread and infection (e.g., ventilation, humidity, toilet plume), and those that are known to enhance wellbeing (e.g. optimal humidity, noise levels, and lighting, greenery, quiet contemplation areas, spaces for exercise and gathering spaces etc.). An automated alarm system will alert facilities managers to hot spots that need mitigation, without adding to their burden of surveillance work, when clusters of perceived risk rise to a pre-determined level, and a navigation tool will use the perceived risk/safety information to provide real-time navigational routes through safer areas on campus. The user will view a real-time campus map, including all outdoor and selected indoor spaces, will select their location, and complete the survey. Users may also aim their smart phones at QR codes posted in selected locations, each linked to the GIS-map, for location-contingent emotion mapping. Users can then view in real-time all survey response results (theirs and others), overlaid on the map as color-coded points that represent perceived feelings of safety and risk and perceived stress (feel safe/unsafe; high/low stress) with respect to perceived health behaviors (e.g., face coverings, social/physical distancing) or sense of stress or wellbeing. Survey results spatially represent the perceptions and reactions of campus-like or building environment occupants in real-time. Knowing this information in real-time can affect campus occupants' movement and behaviors, potentially reducing viral transmission, improving health outcomes, reducing stress, improving wellbeing, and reducing risk and stress in this clustered at-risk population. In that it uses multiple data streams and will be fed into the UArizona central data hub, CyVerse, or into other central data hubs, the invention uses computational, statistical, and mathematical models and artificial intelligence/machine learning for COVID-19 surveillance. The proposed dashboard uses computational modeling to improve the detection, control and prevention of emerging infectious diseases, specifically COVID-19 and provides new prevention approaches. The dashboard will help reduce campus-like or building environment occupants' stress upon re-entering a campus-like environment (e.g. senior living systems, urban areas, shopping malls, office and industrial parks, military bases, jails, prisons, residential treatment facilities), by identifying safer and riskier spaces, or wellbeing spaces on campus and within buildings, and providing safe navigation routes. The use of QR codes will help users within buildings identify their locations anonymously and with minimal subject burden.

The invention comprises a master dashboard, DASH-SAFE/DASH-Well, overlaying subjective perceptions of stress as well as safety and risk, spatially and temporally, on an interactive real-time Geographic Information System (GIS) map of a university campus, but could include any GIS map of any campus-like environment housing clustered buildings and clustered populations (e.g., senior living systems, urban areas, shopping malls, office and industrial parks, military bases, jails, prisons, residential treatment facilities) and could also show only individual buildings and floors within buildings and includes: (1) joint event- and location-contingent experience sampling surveys of perceived safety and risk and wellbeing/comfort; (2) predictive modeling of movement of people through campus or the building over time; and (3) facilities management information for aspects of the built environment that relate to risk of viral spread and infection (e.g., ventilation, humidity, toilet plume) and comfort. An automated alarm system will alert facilities managers to hot spots that need mitigation, without adding to their burden of surveillance work, and a navigation tool will use the risk/safety information to provide real-time navigational routes through safer areas. The dashboard is highly relevant to public health in that it is expected to reduce both stress and risk of viral transmission, and thus help prevent disease, in any at-risk clustered population and will help to mitigate the mental health pandemic that has followed the viral pandemic and is keeping people from re-entering workspaces with confidence and without anxiety and stress.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is an exemplary embodiment of the hardware of the DASH-SAFE/DASH-Well system;

FIG. 2 is an exemplary flow diagram of the software of the DASH-SAFE/DASH-Well system;

FIG. 3 is an exemplary survey that is presented to a user of the system;

FIG. 4 is an exemplary score card scoring system to analyze building design and operations, in accordance with an embodiment of the invention;

FIG. 5A shows an exemplary representation of the output of perceived risk quantification on a computer;

FIG. 5B shows an exemplary representation of the output of perceived risk quantification on a peripheral device;

FIG. 6A shows depictions of how the predictive algorithm exemplarily models the movement of individuals through a food court (left);

FIG. 6B shows a depiction of how the predictive algorithm exemplarily models the movement of individuals through an auditorium (right);

FIG. 7 is a diagram showing the exemplary software components of the invention;

FIG. 8 shows an exemplary consent form, as presented on a peripheral device;

FIG. 9 is a diagram showing exemplary software functionalities of the DASH-SAFE/DASH-Well system;

FIG. 10 is a diagram showing exemplary software components of the DASH-SAFE/DASH-Well system;

FIG. 11 is a diagram showing exemplary software of the DASH-SAFE/DASH-Well system related to risk metrics, predictive modeling, and motion mapping;

FIG. 12 is a chart showing how wireless access points linked to by devices are used to determine the number of occupants in an environment by an exemplary embodiment of the DASH-SAFE/DASH-Well system;

FIG. 13 is a diagram showing how QR codes are used by an exemplary embodiment of the DASH-SAFE/DASH-Well system; and

FIG. 14 is a diagram showing occupancy data on a graphic user interface as a geographic information system (GIS) map.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose. Several preferred embodiments of the invention are described for illustrative purposes, it being understood that the invention may be embodied in other forms not specifically shown in the drawings.

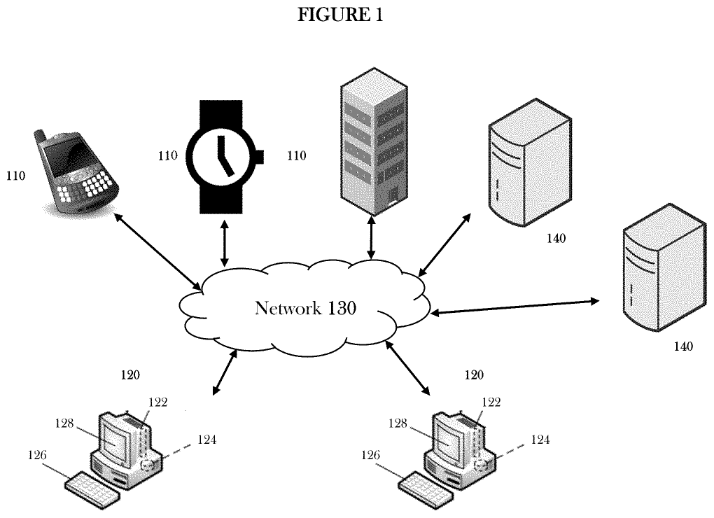

FIG. 1 is an exemplary embodiment of the DASH-SAFE/DASH-Well system. In the exemplary system 100, one or more peripheral devices/locations 110 are connected to one or more computers 120 through a network 130. Examples of peripheral devices/locations 110 include smartphones, networked buildings, wearables devices, GPS devices, infrared sensors, servers with databases that contain a user's personal data, and any other devices that collect data that can be used to collect location and health data that are known in the art. The network 130 may be a wide-area network, like the Internet, or a local area network, like an intranet. Because of the network 130, the physical location of the peripheral devices/locations 110 and the computers 120 has no effect on the functionality of the hardware and software of the invention. Both implementations are described herein, and unless specified, it is contemplated that the peripheral devices/locations 110 and the computers 120 may be in the same or in different physical locations. Communication between the hardware of the system may be accomplished in numerous known ways, for example using network connectivity components such as a modem or Ethernet adapter. The peripheral devices/locations 110 and the computers 120 will both include or be attached to communication equipment. Communications are contemplated as occurring through industry-standard protocols such as HTTP or HTTPS.

Each computer 120 is comprised of a central processing unit 122, a storage medium 124, a user-input device 126, and a display 128. Examples of computers that may be used are: commercially available personal computers, open source computing devices (e.g. Raspberry Pi), commercially available servers, and commercially available portable device (e.g. smartphones, smartwatches, tablets). In one embodiment, each of the peripheral devices/locations 110 and each of the computers 120 of the system may have software related to the system installed on it. In such an embodiment, system data may be stored locally on the networked computers 120 or alternately, on one or more remote servers 140 that are accessible to any of the peripheral devices/locations 110 or the networked computers 120 through a network 130. In alternate embodiments, the software runs as an application on the peripheral devices 110.

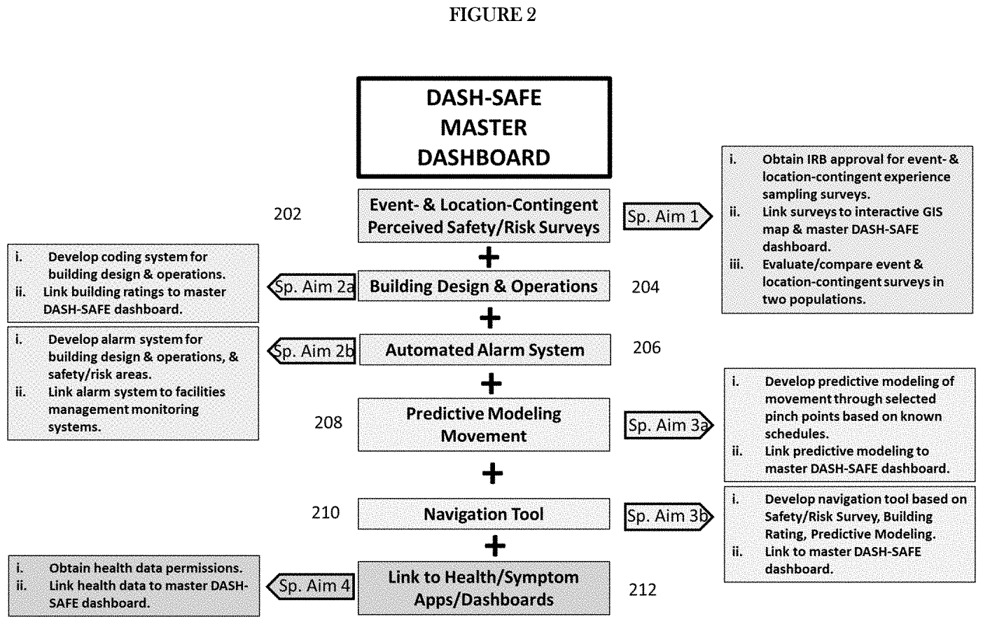

FIG. 2 is an exemplary flow diagram of the software processes performed using the hardware described in FIG. 1 above, in accordance with an embodiment of the invention. The system of the present invention is exemplarily designed to monitor for and identify locations where there is contagion risk, for example, an incident or occurrence that indicates a likelihood of infection or an event that puts individuals at risk of infection. The present invention can be used for a variety of pandemic-related applications, including COVID-19 as well as for post-COVID wellbeing emotion geolocation mapping.

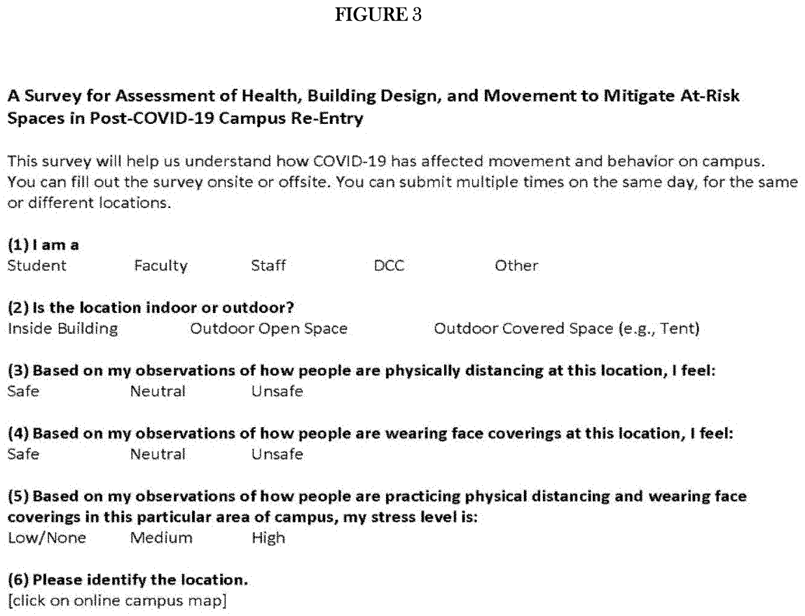

The system commences at “Event- & Location-Contingent Perceived Safety/Risk Surveys” 202, where the system exemplarily collects data from peripheral devices/locations 110. That process can include such steps as: (1) obtaining Institutional Review Board (IRB) approval for event- & location-contingent experience sampling surveys; (2) linking surveys to interactive GIS map & master DASH-SAFE dashboard; and (3) evaluating/comparing event & location-contingent surveys in two populations. An example survey presented to a user of the system is shown at FIG. 3. Event-contingent experience sampling is performed using the surveys when users notice an event that is safe or risky, or a place of comfort or wellbeing and fill out the survey using a peripheral device 110 or computer 120. Location-contingent sampling is performed exemplarily by posting QR codes at the entrance of every building and in other locations throughout the building, and at exits of buildings, or at outdoor connector and potential pinch point locations, which will prompt for experience sampling from users. QR codes may be placed at various locations to facilitate the collection of survey data, such that users may scan the QR codes to access the surveys. In certain embodiments, device location can also be automatically provided if the user allows it. The online survey is accessible through the web link or the QR code. With the location information submitted by survey participants, the dashboard app may be used to read and map survey results.

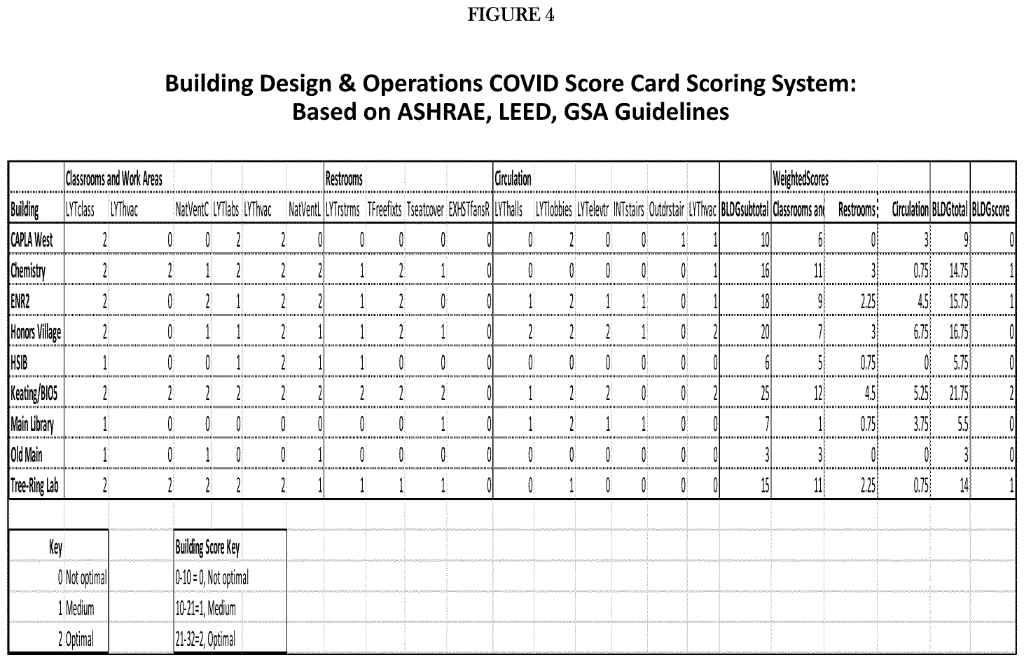

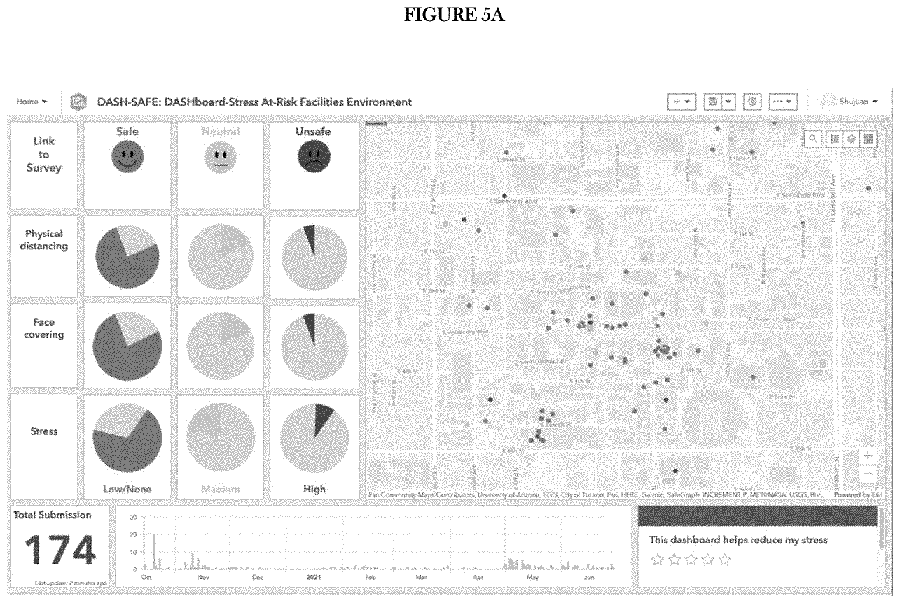

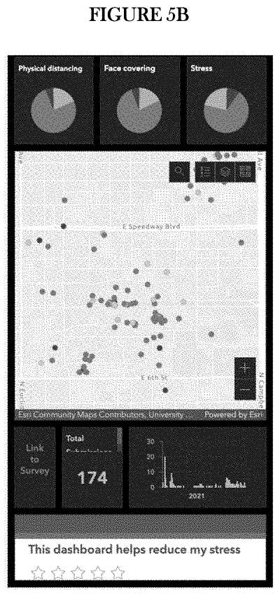

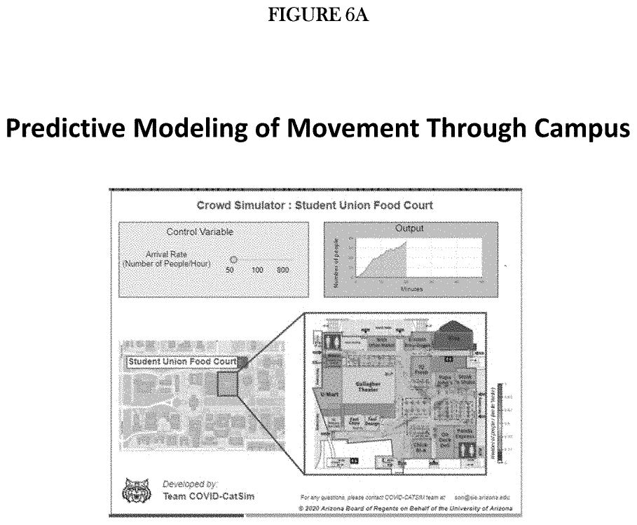

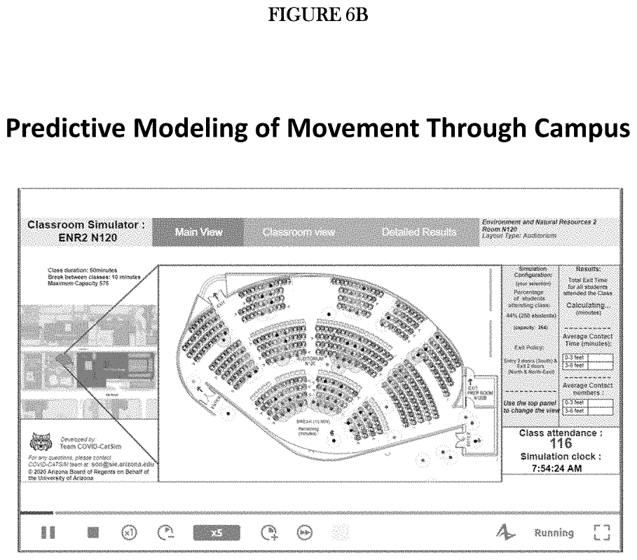

At “Building Design & Operations,” 204, the system collects data from networked buildings 110 and other physical locations and uses that data to rate the building design and operations for safety factors such as overcrowding, airflow, and flow of traffic. Building ratings can be linked to the DASH-SAFE/DASH-Well dashboard of the present invention, and exemplarily quantified using the scoring system shown in FIG. 4. At “Automated Alarm System,” 206, the system implements an alarm system for building design and operations, and for safety/risk areas. Risks may include lack of masks, pinch-points, and crowding, inadequate building design and facilities operations, such as toilet plumes, inadequate ventilation, lack of operable windows. The alarm system may be linked to facilities management monitoring systems, as well as the surveys 202 and output on the peripheral devices 110 or computers 120 of the system, exemplarily as areas of perceived risk. FIG. 5A shows an exemplary representation of that output of perceived risk quantification on a computer 120, while FIG. 5B shows an exemplary representation of that output of perceived risk quantification as on a peripheral device 110. The perceived risk regions may be color-coded to indicate the severity of the perceived risk. At “Predictive Modeling Movement,” 208, the system utilizes a predictive modeling algorithm to determine the movement of individuals. In FIG. 6A, the predictive algorithm is shown exemplarily modeling the movement of individuals through a food court, while in FIG. 6B, the predictive algorithm is shown exemplarily modeling the movement of individuals through an auditorium. In certain embodiments, the predictive algorithm generates individual movement constrained by the space layout (viz. CAD files), and desire or intention of every individual based on a functional area setting customized by the user.

At “Navigation Tool,” 210, the system implements a navigation tool for users based on the safety/risk surveys 202, building rating 204, and predictive modeling 208. The navigation tool is linked to the master DASH-SAFE/DASH-Well dashboard and available on the peripheral devices 110 and computers 120 of the system. At “Link to Health/Symptom Apps/Dashboards,” 212, the system will link to health and other applications that provide information on public health and users of the system, contingent on compliance with HIPAA regulations and other relevant statutes. That health information is linked to the master DASH-SAFE/DASH-Well dashboard and used to determine perceived risk as well as to optimize navigation.

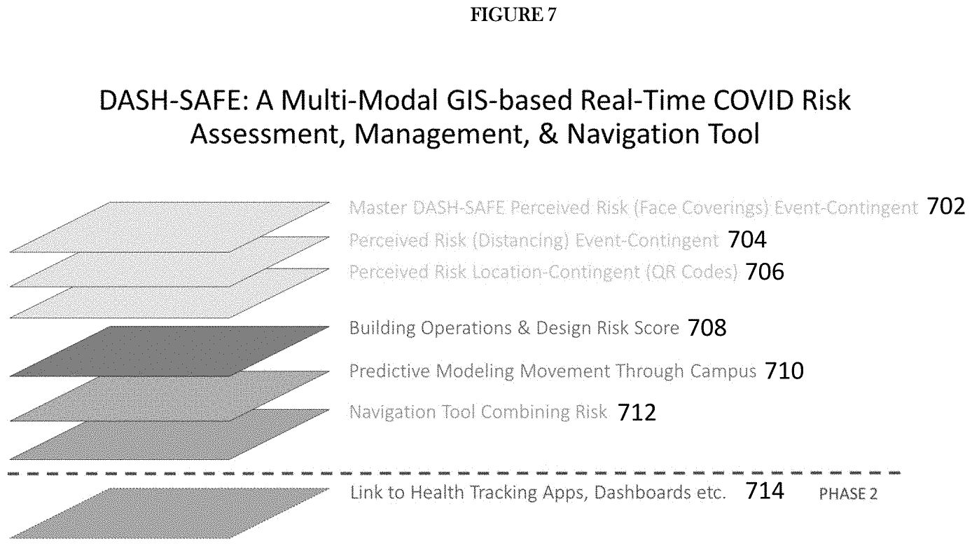

FIG. 7 is a diagram showing the exemplary software components of the invention. The software of the present invention determines the perceived risk at “Master DASH-SAFE Perceived Risk (Face-Coverings) Event-Contingent,” 702, which is a product of the perceived risk determined from event-contingent survey data at “Perceived Risk (Distancing) Event-Contingent” 704 and perceived risk from location-contingent survey data at “Perceived Risk Location Contingent (QR Codes)” 706. As explained with respect to FIG. 2, the software of the present invention also calculates a building operations and design risk at “Building Operations & Design Risk Score” 708. An example of this risk score is shown in FIG. 4. At “Predictive Modeling Movement Through Campus,” 710, the software of the system utilizes a predictive modeling algorithm to determine the movement of individuals through a campus or other campus-like environment. The software of the system analyzes the perceived risk and predictive modeling data at “Navigation Tool Combining Risk” 712, optimizing a safe or relatively safe travel path for users of the system between points in the environment. The analysis is scenario-specific customized. It primarily compares the historic perceived risk, historic modelling risk and the current perceived risk, current modelling risk as a weighted evaluation function and the parameter setting is a per user's focus on the congestion condition of the scenario, the emotion / anxiety feedback of the individual, or the exposure risk related to the disease (e.g. flu) of the individual. The algorithm performs optimization based on a min/max function to balance the potential exposure against the crowdedness, the walking distance, and the walking duration between individual's start point and destination.

In certain embodiments, the software of the present invention, at “Link to Health Tracking Apps, Dashboards etc.” 714, links to health tracking and other applications and dashboards that provide information on public health and users of the system, contingent on compliance with HIPAA regulations and other relevant statutes. That health information is linked to the master DASH-SAFE dashboard and used to determine perceived risk as well as to optimize navigation at step 712.

The innovation of the dashboard is its multi-modal integration of methods and technologies across disciplines, and is thus novel in a broad sense, bringing together methodologies and technologies from multiple disciplines, using real-time GIS technology linked with:

Two different approaches to experience sampling of perceived stress/safety/risk (event- and location-contingent sampling)—thus allowing spatial and temporal visualization of emotional responses to perceived health-related behaviors;

Facilities operation and spatial layout information coupled with a feedback alarm system, to provide immediate spatial and temporal information regarding hot-spots that need to be addressed;

Predictive modeling to demonstrate temporal and spatial movement patterns across campus, thus allowing users to chart safer routes to navigate, related to times of day; and

Health data of viral symptoms and proximity to positive cases, to allow overlaying of cases with facilities and health behavior information to identify potential sources of infection.

The DASH-SAFE/DASH-Well dashboard represents a refinement, improvement, and new application of theoretical concepts, approaches, methodologies, instrumentation, and interventions. Specifically, the dashboard is a new application of GIS technologies to include emotional/perceived stress/safety/risk responses in real-time and place. It puts into practice in real-time and real-place, a long predicted but not yet broadly methodologically realized theoretical construct for location-contingent experience sampling, “context-aware experience sampling”, developed by Stephen Intille at MIT in the early 2000s. Context-aware experience sampling renders experience sampling more efficient and effective by prompting participants only when they are located in contexts of interest, for example, specific locations. It is a new preventive intervention that can be applied at any scale to prevent the spread of disease and stress.

Data Sharing Plan DASH-SAFE Dashboard:

The data, results, and analyses of this invention will be rapidly disseminated and shared with the broader scientific community, using existing public repositories whenever possible, as a foundation for further study. The data will be fed into the University of Arizona's Data Science Institute's central data managing system, CyVerse, an open science workspace for collaborative data-driven discovery http://www.cyberse.org/ as described below.

Data Collection, Handling, Sharing and Reporting:

Data collection: For each participant in the event-contingent pilot, his/her data from the DASH-SAFE dashboard will be stored in delimited files that will be ingested into an open source time series database influxDB, and stored in the CyVerse data store. No information specific to a person's ID will be collected and also compliance with the IRB protocol will be ensured.

### EXAMPLE 1

### Implementation of the Prototype Invention at University of Arizona

The present invention is advantageous because it is multifnodal and addresses a high-risk clustered population—i.e., students and indeed any occupants on campus. Campuses are clearly high-risk clustered populations. as evidenced by the fact that many universities have opted to begin and remain completely online, and some opened briefly and then closed within a week or two, due to clustered COVID-19 outbreaks. The University of Arizona has chosen a hybrid approach combining in-person and online classes. To reduce risks of infection spread as much as possible, campus occupancy is being informed by dashboards and apps being developed, which will coordinate with ours. The situation is urgent and we are on a very short timeline to develop the dashboard at the request of the University President, to provide a data-driven basis for decision-making for daily briefings in the University of Arizona Incident Command System, led by former Surgeon General Richard Carrnona, Distinguished Professor at the University of Arizona's Mel and Enid Zuckerman College of Public Health. Another aspect of our invention's program fit is the ability of the University of Arizona Data Science Institute's CyVerse data hub to both automatically upload our data set to the NIH RADx-rad Data Coordination Center hub, as well as to provide ari open access data platform.

Environment

The scientific environment in which the work will be carried out will contribute greatly to the probability of the invention's success. The University of Arizona has an infrastructure that supports and encourages inter-disciplinary, multi-College research, as evidenced by the long history of members of this team working together on other projects and indeed the seed funding for this invention, as members of the Institute on Place Wellbeing & Performance (IPWP). There are many ways that the institution formally supports these multi-disciplinary, multi-College teams, through a cross-cutting infrastructure that supports centers and institutes composed of at least three units. Each member of an institute or center must also have a primary appointment in a College. The IPWP, which Dr. Sternberg established upon coming to the University of Arizona, is an example, linking the College of Medicine, The Andrew Weil Center for Integrative Medicine, the College of Architecture, Planning and Landscape Architecture, the College of Engineering and others. Another example is the BIO5 Institute https://bio5.org/, which encourages multi-College and interdisciplinary research between the five health science colleges and engineering, by providing a robust series of seminars, core services, such as statistics and a prototyping lab. Importantly and relevant to this invention, under the BIO5 institute umbrella are the Data Science Institute, https://datascience.arizona.edu/ which provides computational and informatic support. The Data Science Institute immediately at the start of the COVID-19 pandemic in March, launched AZCOVIDTEXT https://azcovidtxt.org/, a platform for COVID-19 support, to track, map and contain COVID positive cases across Southern Arizona. This is one of the resources to which this invention proposes to link our DASH-SAFE dashboard. Also linked to B105 is the linked resource, CyVerse https://www.cyverse.org/—an open science workspace for collaborative data-driven discovery.

Th CyVerse resource will contribute greatly to the computational and open data sharing components of this invention and will provide a seamless mechanism for continuously sharing the outcomes and data from the dashboard with the proposed RADx DCC. Other core services that are available and relevant to this invention are the Arizona Cancer Center's Behavioral Measurement and Interventions Shared Resource https://cancercenter.arizona.edu/researchs/shared-resources/behavioral-measurement-and-interventions which provides independent evaluation of behavioral intervention studies and will be carrying out the evaluation component of the DASH-SAFE dashboard's effectiveness and the two experience sampling pilot studies. Another core service relevant to this invention is UX@UA https://uxua.arizona.edu/ which will provide user experience advice and evaluation.

Human Subjects Plan DASH-SAFE Dashboard:

While the main aim of the DASH-SAFE/DASH-Well dashboard is to identify risky and safe locations on campus for navigation and mitigation, two studies involving human subjects were carried out, as outlined below: an event-contingent and location-contingent program, to compare their accuracy and effectiveness in identifying such areas, and to assess usability of both survey approaches to determine which provides more actionable information.

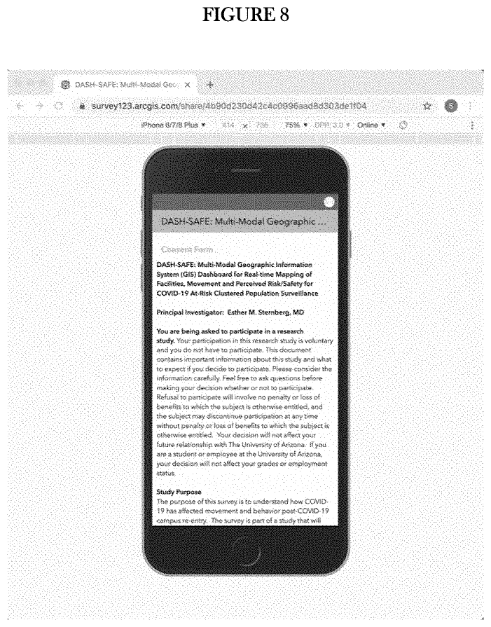

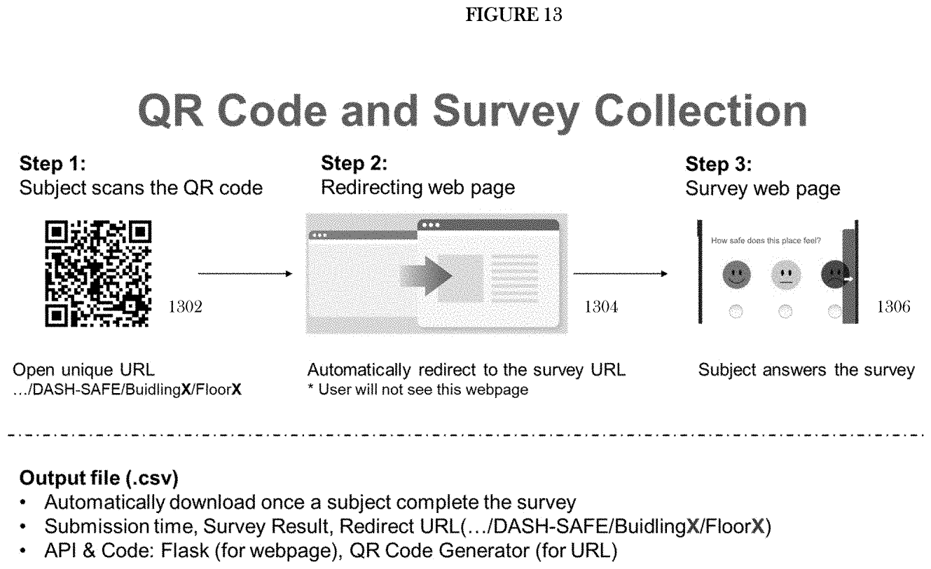

In the event-contingent program, a cohort of student ambassadors will be trained to recognize both particularly safe and particularly risky areas, and to respond to the location survey on the dashboard when they identify them. In the location-contingent survey, individuals will aim their smart phones at QR codes posted around campus, at entrances and exits to buildings and outdoors, and will click on smiley/neutral or frowny face to provide their perception of safety and risk. QR code usage is further described with respect to FIG. 13 below. Before taking the survey for the first time, the individuals will sign a consent in which they will identify whether they are a student, faculty or other. An example consent form is shown in FIG. 8.

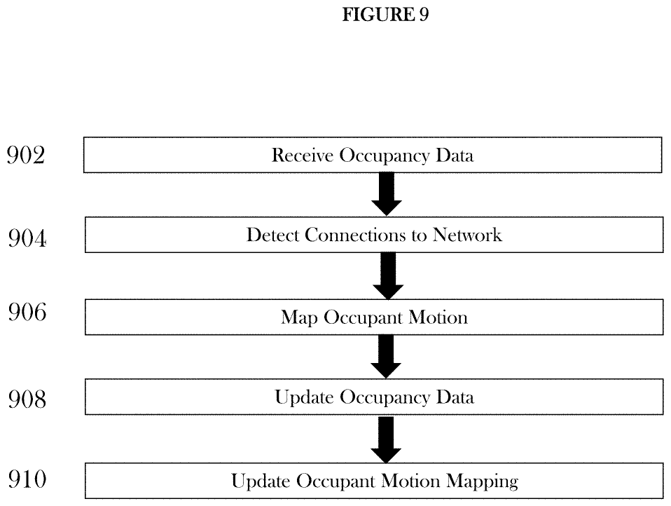

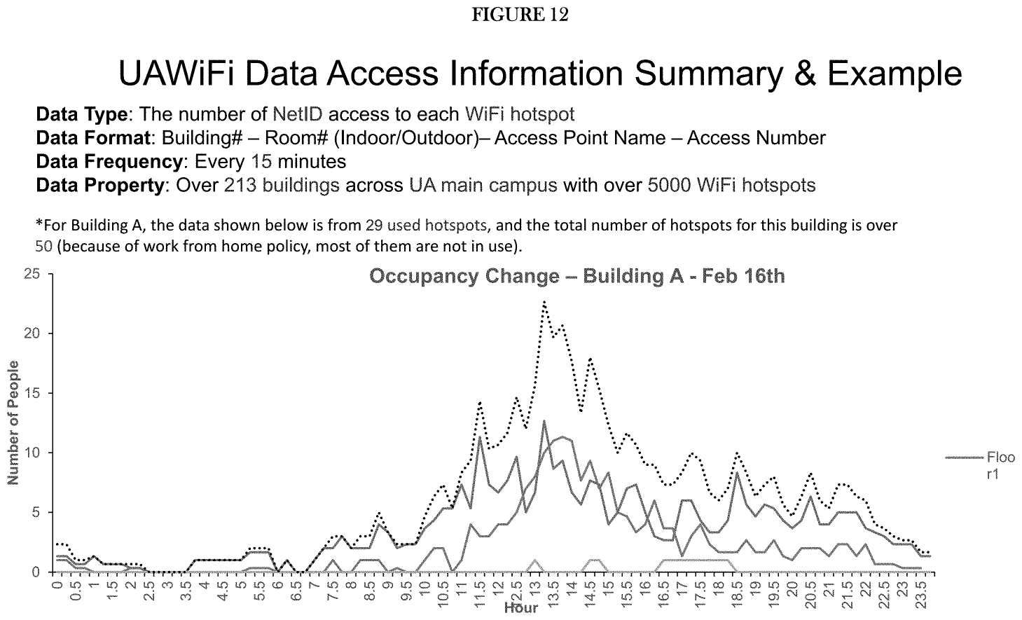

FIG. 9 is a diagram showing exemplary software functionalities of the DASH-SAFE system. The process commences at “Receive Occupancy Data” 902, where the system of the present invention receives data associated with occupants in a particular closed environment. The data may be collected in real-time or at intervals. In certain embodiments, the intervals may be 15 minutes, 30 minutes, 1 hour, 2 hours, or 4 hours. At “Detect Connections to Network” 904, the software of the present invention monitors for device connections to the network (i.e. Internet). The system receives notification any time a device attempts to connect to the network and may also receive an identifier associated with that device, for example, a Net-ID. The identifier is preferably unique and is used to ensure that each device and the individual associated with that device is tracked discretely and not duplicatively counted. An example of this process is shown in FIG. 12 below.

Based on the collected occupancy data and the detected network connections, at “Map Occupant Motion” 906, the system creates a visual representation of the occupants in the environment of interest, exemplarily applying the predictive modeling described with respect to FIG. 6. Based on the current occupancy and the flow-in and flow-out data of the same building at the same day of the week, the future flow-in and flow-out individual amount in the next 15 minutes or next hour can be estimated. Then, based on the Network Analyst function, the software firstly collected the available destinations within the X-minutes walking zone around the start-up point, then built a set of the available routes among these buildings, combining the different walking speed on different routes, it is able to map the motion with highest probability and show the most occupied or crowd pathways in the motion range. The Network Analyst function is used to dynamically model the realistic network conditions at different time periods of a day. The network dataset includes features as origin and destination areas or points (academic buildings, food courts, bookstores, restaurants, . . . ), lines (routes) and turns, and the connectivity of these features. The Network Analyst function is used to create origin-destination cost matrices of which the network cost is expressed with mathematical expressions of the distance, time, penalty, and reward. In this case, the value of the risk matrix of the building was added to the road or pathway near the building as penalty values. The origin-destination cost matrix contains the network impedance between every two intersections from the origin to the destination; therefore, it not only shows the shortest or optimistic routes of an individual but also shows the congestion estimation of each route by combining the cost matrix of different moving individuals.

At “Update Occupancy Data” 908, the system receives updated data associated with the occupants of the environment, either in real-time or at the exemplary intervals discussed above. At “Update Occupant Motion Mapping” 910, the visual representation of the occupants in the environment of interest is updated to reflect their most recent locations in the environment. The occupancy data will provide users information about how close the building is to maximal safe occupancy for reduction of viral spread, so that they can choose whether or not to enter it and at what times of day they might enter it.

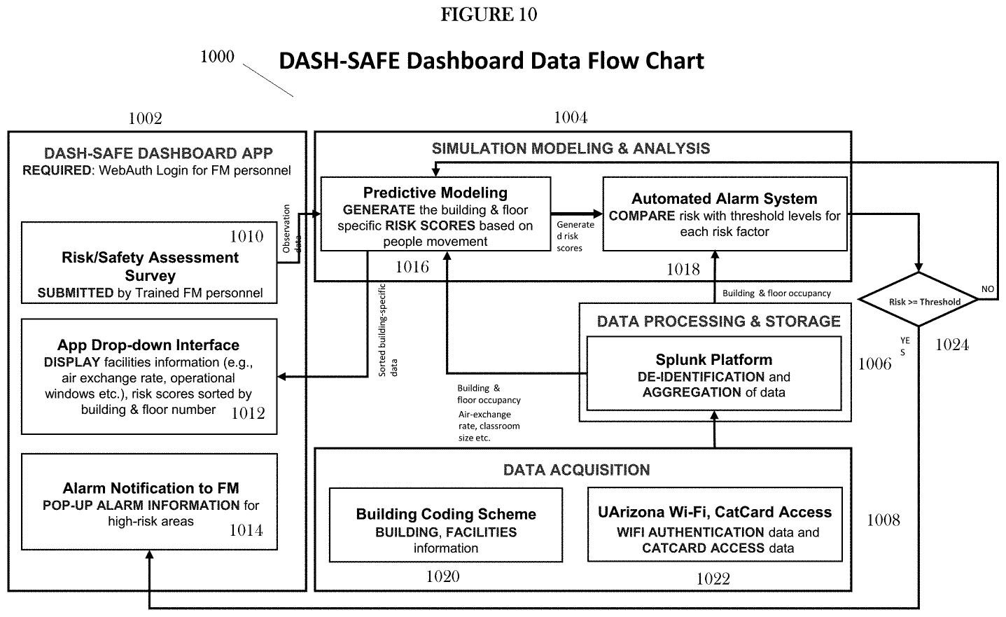

FIG. 10 is a diagram showing exemplary software components of the DASH-SAFE system, specifically showing the various modules that comprise the system and their interactions. The system 1000 is comprised of the dashboard application 1002, the simulation, modeling, and analysis module 1004, and the data processing and storage module 1006, and the data acquisition module 1008.

The dashboard application 1002 is designed to operate on peripheral devices/locations 110 and the computers 120 of the system 1000. The dashboard application 1002 is further comprised of the risk/safety assessment survey module 1010, the app dropdown interface 1012, and the alarm notification to FM module 1014. In certain embodiments, the dashboard application 1002 requires web authentication login for individuals prior to use. The risk/safety assessment survey module 1010 is responsible for allowing users to take an event-contingent and location-contingent experience sampling survey of perceived safety and risk. The app dropdown interface 1012 displays, through a graphic user interface, facilities information (e.g., air exchange rate, operational windows etc.), risk scores, exemplarily sorted by building & floor number. The alarm notification to FM module 1014 displays, using a graphic user interface, information that identifies high-risk areas in the environment that should be avoided. In certain embodiments, the information is displayed using a pop-up notification on the graphic user interface.

The simulation modeling and analysis module 1004 is further comprised of the predictive modeling module 1016 and the automated alarm system module 1018. The simulation modeling and analysis module 1004 connects to the dashboard application 1002 and the data processing and storage module 1006. The data processing and storage module 1006 is responsible for the de-identification and aggregation of the data collected by the system 1000. In certain embodiments, the processing and storage module 1006 runs on a Splunk platform. The data processing and storage module 1006 is connected to the data acquisition module 1008. The data acquisition module 1008 is further comprised of the building coding scheme module 1020 and the access card module 1022.

The predictive modeling module 1016 generates risk scores for locations in the environment (e.g. buildings and floors) based on the movement of people. Total risk is a weighted function comprised of every individual's movements, with the average data as the maximum likelihood estimation and also the upper boundary as an estimation of an extreme condition. The predictive modeling module 1016 receives observation data from the from the risk/safety assessment survey module 1012 and building and floor occupancy data, along with the air-exchange rate, room size, and other such data from the data processing and storage module 1006. The threshold may be user-defined, based on the designed occupancy limits of the environment. The predictive modeling module 1016 transmits data such as sorted building-specific data and risk scores to the app drop-down interface, which uses that data to display facilities information and risk scores, which may be sorted by building and floor number in certain embodiments.

The automated alarm system module 1018 compares risk scores against predetermined threshold levels for each risk factor. The automated alarm system module 1018 receives data such as building and floor occupancy numbers from the data processing and storage module 1006 and receives the generated risk scores from the predictive modeling module 1016. Using that data, the automated alarm system module 1018 determines whether the risk score exceeds a predetermined threshold 1024. If the risk score is higher than the predetermined threshold, then the automated alarm system module 1018 transmits a signal to the alarm notification to FM module 1014, which notifies the user of the high-risk environment. If the risk score does not exceed the predetermined threshold, then the automated alarm system module 1018 transmits a signal to the predictive modeling module 1016 to continue generating risk scores using updated data.

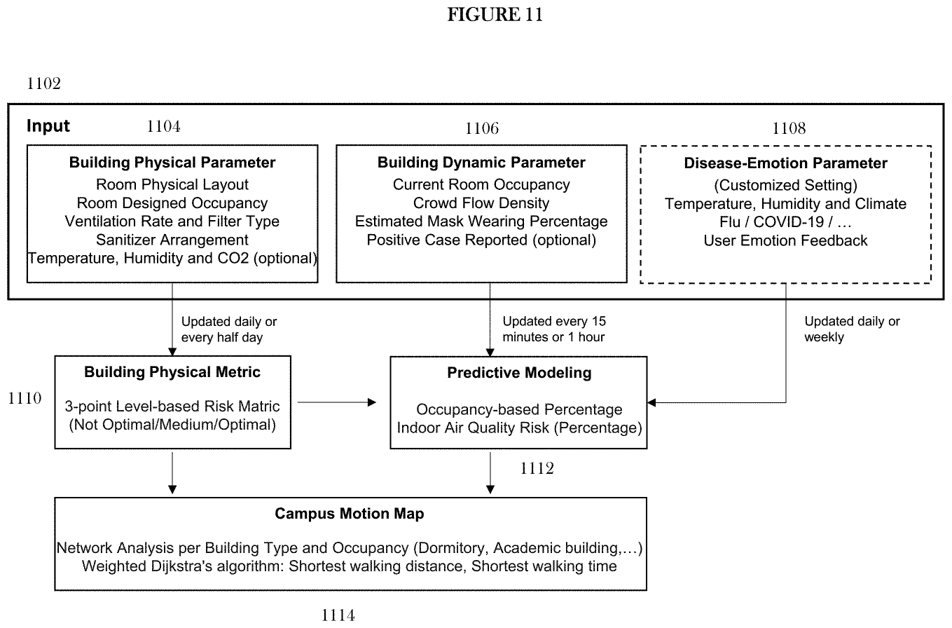

FIG. 11 is a diagram showing exemplary software of the DASH-SAFE system related to risk metrics, predictive modeling, and motion mapping. The system collects a number of inputs 1102 in order to perform its risk metrics, predictive modeling, and motion mapping functionalities. Inputs 1102 comprise one or more building physical parameters 1104, one or more building dynamic parameters 1106, and one or more disease-emotion parameters 1108. The one or more building physical parameters 1104 may include a room's physical layout, a room's designed occupancy limits, ventilation rates and filter type, sanitizer arrangement, room temperature, room humidity and room CO₂ levels. The one or more building dynamic parameters 1106 may include current room occupancy numbers, the crowd flow density, estimated mask-wearer percentage, and positive cases reported. The crowd flow density is exemplarily calculated as D(Crowd Flow)=F(Space Layout, Duration, Occupancy), where the crowd flow density (left) is a function of the space layout, duration, and occupancy. As the current occupancy data is available, and it is possible to estimate the flow-in, flow-out amount, the crow flow density may be calculated via available routes / space related to space layout over a predetermined period of time, for example, in the next fifteen minutes. The one or more disease-emotion parameters 1108 may include the ambient temperature of the environment, the environmental humidity, the disease type being monitored (e.g. flu, COVID-19), and user emotional feedback, which may be collected from the user's peripheral devices.

The one or more building physical parameters 1104 are used to calculate a building physical metric 1110, for example, as shown in FIG. 4. In certain embodiments, the building physical metric 1110 may be a 3-point level-based risk metric, where the metric has three states: “not optimal,” “medium,” and “optimal.” The building physical metric 1110 may be updated in real-time or periodically, for example, once or twice daily.

The one or more building dynamic parameters 1106 and one or more disease-emotion parameters 1108 are used for predictive modeling 1112 per user customization, which results in a determination of the occupancy-based percentage indoor air quality risk, a risk percentage for an indoor environment.

The building physical metric 1110 and predictive modeling 1112 are used by the system to output a motion map 1114 that shows the use of wireless networks per building type, building occupancy, and the shortest walking distance and times to travel safely between locations in the environment being analyzed by the system. Based on the current occupancy and the flow-in and flow-out data of the same building at the same day of the week, the future flow-in and flow-out individual amount can be estimated for a predetermined period of time, for example, in the next 15 minutes or next hour. Then, based on the Network Analyst function, the software firstly collects the available destinations within the X-minutes walking zone around the start-up point, then built a set of the available routes among these buildings, combining the different walking speed on different routes, it is able to map the motion with highest probability and show the most occupied or crowd pathways in the motion range. The building risk metric provides a penalty when the software calculates the shortest walking distance and time score to determine the routes with highest probability for an individual. In certain embodiments, a weighted Dijkstra's algorithm is used to determine the shortest walking distance and times to travel safely between locations.

FIG. 12 is a chart showing how “hotspots” used by devices to wirelessly connect to a network are used to determine the number of occupants in an environment (Building A). As shown in FIG. 12, the system of the present invention collects data related to the number of NetIDs accessing each hotspot. The data is comprised of the building number, the room number, whether that room is indoors or outdoors, the access point (hotspot) name, and the number of NetIDs accessing each access point. In this embodiment, the data is updated every fifteen minutes, and may be collected in an array of buildings and environments, as contemplated by one of ordinary skill in the art.

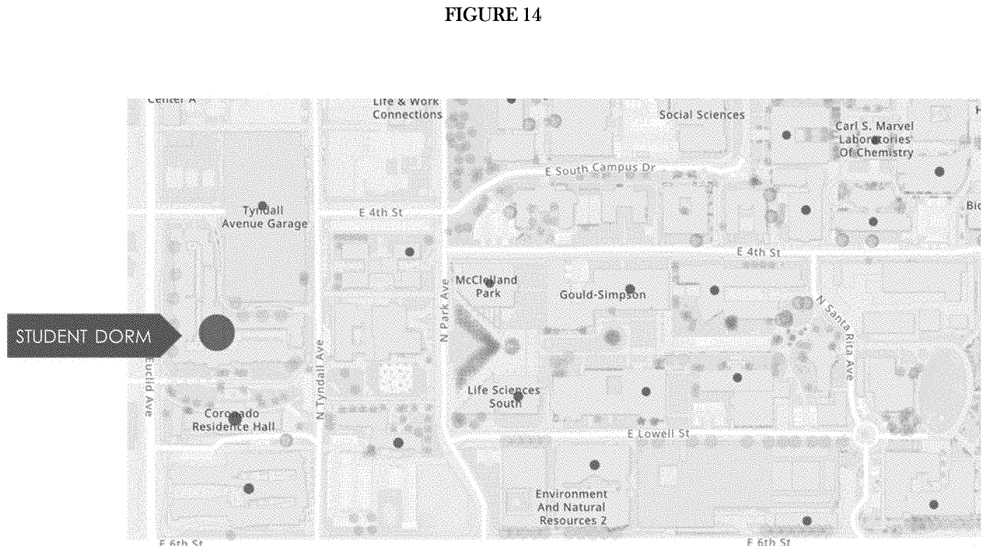

FIG. 13 is a diagram showing how QR codes are used by an exemplary embodiment of the DASH-SAFE/DASH-Well system. At step 1302, an individual is able to scan a QR code using his or her peripheral device. QR codes may be placed at various locations to facilitate the collection of survey data, such that users may scan the QR codes to access the surveys. In certain embodiments, device location can also be automatically provided if the user allows it. At step 1304, the user's peripheral device is directed to an online survey, which is located at a web address (e.g. a URL) that is associated with or the QR code. At step 1306, the user may input answers to the survey. With the location information submitted by survey participants, the dashboard app may be used to read and map survey results, as exemplarily shown in FIG. 14.

### EXAMPLE 1

### Event-Contingent Program

(a) Population: Stfudent ambassadors, part of the University of Arizona T3 Re-Entry plan. These comprise x males and y females, ages a-z.

(b) Recruitment plan: A briefing will be presented regarding the survey to the Incident Command System leadership and committee for their buy-in and will work closely with the ICS to recruit and train the ambassadors.

(b) Availability of population and past successes in recruitment: The population is highly available, as they are an important part of the T3 Re-Entry program team.

Location: The program is carried out on the University of Arizona campus and in selected buildings across campus.

Infection Control: All student ambassadors wear PPE (masks and gloves) when interacting with each participant at all stages of the program. Their primary role is to hand out masks and viral safety information as they move about campus, and wear masks at all times. The University of Arizona has mandatory precautions in place for infection control including restricted entrances, mandatory temperature checks for everyone entering the facility, available SARS-CoV-19 testing, SARS-CoV-19 antibody testing, and all necessary medical, surgical, and trauma services.

Potential Risks: There are no known risks to taking a survey on one's own smartphone, as there are no personal interactions that will take place. There may be a risk that student ambassadors may be viewed by the campus occupants as informers, however adding the DASH-SAFE dashboard to their duties does not increase this risk. Furthermore, this perception will be diminished by the consent form emphasizing that their privacy will be protected, that there will not be tracking of their phones with GPS and will not be collecting personal identifiers. There is the risk that the DASH-SAFE dashboard may add to their subject burden by requesting them to take the time to take the survey. However, we anticipate that on the contrary, by providing an automated way to identify safe and risky spaces, the ambassadors' task load will actually be lightened.

### EXAMPLE 2

### Location-Contingent Program

Population: All University of Arizona campus occupants.

Recruitment plan: A marketing plan will be developed to disseminate information about the DASH-SAFE dashboard including using social media, listservs, posters and postings of QR codes on information LCD screens across campus and on “Talk-Back” boards at libraries, and campus-wide zoom symposia presentations.

Availability of population and past successes in recruitment: The population is highly available as it is comprised of all campus occupants.

Location: The program was carried out on the University of Arizona campus both outdoors and at entrances and exits of selected buildings across campus.

(a) Infection Control: As this is a touchless system of simply aiming the smart phone at the QR codes, infection control does not apply other than the standard University of Arizona mandatory precautions in place for infection control including restricted entrances, mandatory temperature checks for everyone entering the facility, available SARS-CoV-19 testing, SARS-CoV-19 antibody testing, and all necessary medical, surgical, and trauma services. The program will not interfere with these precautions and, on the contrary, seeks to identify areas where the precautions are particularly well or particularly not adhered to.

(b) Potential Risks: There are no known risks that are specifically associated with remote measurement of QR codes.

Adequacy of protection against risks for both Event-contingent and location-contingent studies:

(a) Recruitment and informed consent: A written informed consent form, provided by a link to through RedCap-linked Survey 123 on Arc-GIS, will be required before a user can move to the next step of taking the survey. The consent uses language understandable to the participant or the surrogate, and has been reviewed by and approved by the IRB. The entire pilot protocol as well as potential risks to potential participants are described on the online consent. All potential subjects will be specifically informed that their decision not to participate in the pilot will in no way compromise their health. All experimental protocols consent forms will be reviewed and approved by the Institutional Review Board (IRB) of the University of Arizona. Research will be performed only after the approval of the IRB has been obtained.

(a) Protection against risk: Participant safety and privacy are a top priority in our pilot and takes precedence over retention at all times.

(b) Detailed information regarding IT-data security protection: Initially created for the National Children's Study, the University of Arizona and the Arizona Respiratory Center have created a secure FISMA-compliant environment for the collection and management of data. It is housed in a limited-access, secure data center located on the first floor of the University of Arizona, College of Medicine, in Tucson, Arizona. Access to the server room is granted by the Chief Information Officer. Servers are primarily based on Microsoft Windows Server 2008. Servers are virtualized on VMware VSphere platform which allows us to easily and effectively segregate testing and production environments. Dell R510 servers provide for high availability systems and replication of information system components. ReadyNAS 3200 is used as shared storage and allows for easy migration of virtual machines in case of systems failure. An additional system will be located in a secure alternate off-site facility and used for full system backups and system replication. All system hardware components include Enterprise levels of Hardware and OS Support. A dedicated Juniper SSG320 firewall connects to the interne via the UA Campus network and segregates the secured VLAN system components from the campus network. The Juniper SSG320 firewall provides stateful packet inspection and policy-based access restriction; remote access VPN service; logging, as well as Unified Threat Management (UTM) which includes Intrusion Detection capabilities. BitLocker Drive Encryption is a full disk encryption feature included with Windows 7 Operating Systems, as well as the Windows Server 2008 and Windows Server 2008 R2 server platforms. It is used to protect data by providing encryption for entire volumes. By default, it uses the AES encryption algorithm in CBC mode with a 128-bit key. BitLocker provides two-factor authentication and disk encryption of all remote end-stations. Sophos Endpoint Security and Control 10.2 is used on all remote and local end-stations as well as servers. Sophos Endpoint Security and Control protects all of our endpoints. It is a single, integrated solution that secures systems and data against sophisticated malware, zero-day attacks, noncompliant systems, and unauthorized devices. It also protects lost or stolen devices by blocking non-permitted access to our business-critical systems and sensitive data. A Cisco ASA 5505 is used to create secure SSL VPN connections for the information system. The Cisco ASA 5505 includes full support for IPsec and SSL VPN endpoints, providing highly encrypted tunnels for office to office and remote user to office connections. All of the current participant information will also be kept and daily updated in an encrypted server drive space of the University of Arizona.

(a) Security Policy: A robust security policy has been adopted that is fully compliant with the Privacy Act of 1974, the HIPAA Privacy Rule, and federal statutes for the protection of subject information. These policies are the most rigorous possible without compromising our research mission. Issues addressed are devoted to the roles of research personnel; location of the data; organization of files; authorization and authentication procedures; management of identifiers; approved uses of the data; IRB approvals; informed consent, HIPAA authorization, or their waivers; re-contacting participants and re-use of data; protection from loss; security of workstations; data transfers; monitoring user logs; archiving and retention of files; record-keeping; and reporting requirements.

(b) Data and Safety Monitoring Plan: Data monitoring plan: Each subject will be assigned an identifying number to maintain confidentiality. Records will be stored in a database format that can be read by a standard statistical package. Forms will be formatted to facilitate accurate entry and editing, and programs will be written to allow range checks at entry with illegal values not accepted. Specific missing codes will identify non-obtainable data due to missed visits or skipped questions. The research coordinator will enter the data and the PI will proofread the forms. The data manager and the coordinator will perform weekly data verification and protocol compliance checks. Safety monitoring plan: Adverse event reporting will be monitored in accordance with the guidelines and regulations of the University of Arizona IRB. This pilot does not require a data safety monitoring board (DSMB). Any serious and/or unanticipated problems will be reported immediately to the University of Arizona Institutional Review Board (IRB) and the pilot sponsor as required. The PI and coordinator will monitor adherence to the protocol and data quality standards and monitor for participant safety and evidence for adverse or beneficial effects. The principal investigator will prepare annual report for review by the IRB. The reports will include: screening and baseline data, efficacy data, safety and adverse data, subject enrollment data, and dropout data (number, reason, and at what phase).

Definition of adverse events: An adverse event (AE) is any unfavorable and unintended sign, symptom, or disease temporally associated with the participation in the pilot, whether or not considered related to pilot. (a) AEs will include: 1. Changes in the general condition of the participant; 2. Subjective symptoms offered by or elicited from the participant; 3. Signs observed by the investigator or personnel; 4. All concurrent disease including any change in severity or frequency of pre- existing disease. (b) Serious AE is one that: 1. Results in death; 2. Is an immediate threat to life; 3. Results in permanent disability. In addition, a serious AE is one that is judged by the investigator to be an important or medically significant event. Causality assessments of AEs: For all AEs, the investigator will provide an assessment of causal relationship to pilot participation. Appropriate forms will be used for this purpose and filed in the case report forms and submitted to the IRB and R&DC for review. They will be classified as related, possibly related, and not related. The severity will be adjudged as being mild, moderate, or severe.

The foregoing description and drawings should be considered as illustrative only of the principles of the invention. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

1. A personal risk-assessment and risk-management, and wellbeing emotion-mapping navigation system comprising one or more servers, wherein the servers:
receive location-based survey data from one or more peripheral devices;
receive event-based survey data from one or more peripheral devices;
receive building design and operations data;
apply predictive modeling to quantify perceived risk of disease;
map motion of individuals associated with the one or more peripheral devices into a visual representation; and
output, to a graphical user interface at the one or more peripheral devices, perceived risk data and the visual representation of the mapped motion of individuals.

2. The system of claim 1, wherein the perceived risk data is color- or shape-coded.

3. The system of claim 1, wherein the servers receive health data from one or more applications.

4. The system of claim 1, wherein the one or more servers further output an alarm based on an increased level of perceived risk of disease.

5. The system of claim 1, wherein the one or more servers further provide a navigation tool to the graphical user interface at the one or more peripheral devices.

6. The system of claim 1, wherein the location-based survey data is comprised of occupancy data and network connection data.

7. The system of claim 6, wherein the occupancy data is collected in real-time or at time intervals.

8. The system of claim 1, wherein the server further receives updated location-based survey data from QR coded signs posted throughout locations of interest.

9. The system of claim 8, wherein the server further updates the visual representation of the mapped motion of individuals based on the updated location-based survey data.

10. The system of claim 1, wherein the location-based survey data is comprised of a unique identifier associated with each of the one or more peripheral devices.

11. A computer-implemented method of personal risk-assessment and risk-management and wellbeing emotion mapping navigation, comprising the steps of:
receiving location-based survey data from one or more peripheral devices;
receiving event-based survey data from one or more peripheral devices;
receiving building design and operations data;
applying predictive modeling to quantify perceived risk of disease;
mapping motion of individuals associated with the one or more peripheral devices into a visual representation; and
outputting, to a graphical user interface at the one or more peripheral devices, perceived risk or wellbeing data and the visual representation of the mapped motion of individuals.

12. The computer-implemented method of claim 11, wherein the perceived risk or wellbeing data is color- or shape-coded.

13. The computer-implemented method of claim 11, further comprising health data from one or more applications.

14. The computer-implemented method of claim 11, further comprising outputting an alarm based on an increased level of perceived risk of disease.

15. The computer-implemented method of claim 11, further comprising providing a navigation tool to the graphical user interface at the one or more peripheral devices.

16. The computer-implemented method of claim 11, wherein the location-based survey data is comprised of occupancy data and network connection data.

17. The computer-implemented method of claim 16, wherein the occupancy data is collected in real-time or at time intervals.

18. The computer-implemented method of claim 11, further comprising receiving updated location-based survey data.

19. The computer-implemented method of claim 18, further comprising updating the visual representation of the mapped motion of individuals based on the updated location-based survey data.

20. The computer-implemented method of claim 11, wherein the location-based survey data is comprised of a unique identifier associated with each of the one or more peripheral devices.
